SEQUENCE LISTING (11 sequences)

SEQ ID NO: 1 - DNA, 41 bp, Artificial sequence
  Synthetic
ggcgcggccgcatggggcccagcttagacttcgccttgtct

SEQ ID NO: 2 - DNA, 41 bp, Artificial sequence
  Synthetic
cgcctcgagttaaaataactccagccaatttcttctcaagt

SEQ ID NO: 3 - DNA, 16 bp, Artificial sequence
  Synthetic
gcggccgctctagacc

SEQ ID NO: 4 - DNA, 11 bp, Artificial sequence
  Synthetic
tcgacaagctt

SEQ ID NO: 5 - DNA, 30 bp, Artificial sequence
  Synthetic
ggcctagggtattagtcatcgctattacca

SEQ ID NO: 6 - DNA, 30 bp, Artificial sequence
  Synthetic
ggcctaggtccccagcatgcctgctattgt

SEQ ID NO: 7 - DNA, 48 bp, Artificial sequence
  Synthetic
ctctagaagcttcctaaaaaaccgccaccatgggttcgcaagtgtcta

SEQ ID NO: 8 - DNA, 22 bp, Artificial sequence
  Synthetic
gcgtgactgcctgcctaagacc

SEQ ID NO: 9 - DNA, 2589 bp, Enterovirus 71
  CDS (1)..(2589)
atgggttcgcaagtgtctacacagcgctccggttctcacgaaaactcaaactcagccactgagggttctaccataaactacaccaccattaattactacaaagactcctatgctgccacagcaggcaaacagagtctcaagcaggatccagacaagtttgcaaatcctgttaaagacatcttcactgaaatggcagcgccactgaagtccccatccgctgaggcatgtggatacagtgatcgagtggcgcaattaactattggcaactccaccatcaccacgcaagaagcggctaatatcatagtcggttatggtgagtggccttcctactgctcagattctgacgctacagcagtggataaaccaacgcgcccggatgtttcagtgaacaggttttacacattggacactaaattgtgggagaaatcgtccaagggatggtactggaagttcccggatgtgttaactgaaactggggtttttgggcaaaatgcacaattccactacctctaccgatcagggttctgcatccacgtgcagtgcaatgccagtaaattccaccaaggagcactcctagtcgctgtcctaccagagtatgtcattgggacagtggcaggcggtacagggacggaagatacccaccccccttacaagcagactcaacccggcgccgatggcttcgagttgcaacacccgtacgtgcttgatgctggcatcccaatatcacagttaacagtgtgcccacaccagtggattaatttgaggaccaacaactgtgctacaataatagtgccatacattaacgcactgccttttgattctgccttgaaccattgcaactttggcctgttggttgtgcctattagcccactagactacgaccaaggagcgacgccagtaatccctataactatcacattggccccaatgtgttctgaattcgcaggtcttaggcaggcagtcacgcaagggttccccaccgagctaaaacctggcacaaatcaatttttaaccaccgatgatggcgtttcagcacctattctaccgaacttccaccccaccccgtgtatccacatacctggtgaagttaggaacttgctagagttatgccaggtggagaccattctggaggttaacaatgtgcccacgaatgccactagcttaatggagagactgcgcttcccggtctcagcacaagcagggaaaggtgagctgtgtgcggtgtttagagccgatcctgggcgaaatggaccatggcaatccaccttactgggtcagttgtgcgggtactacacccaatggtcaggatcattggaagtcaccttcatgtttactggatccttcatggctaccggcaagatgctcatagcctatacaccgccaggaggtcctctgcccaaggaccgggcgaccgccatgttgggcacgcacgtcatctgggattttgggctgcaatcgtctgttacccttgtaataccatggatcagcaacactcattatagagcacatgcccgagatggagtgtttgactactacaccacagggttagtcagtatatggtatcagacaaattacgtggttccaatcggtgcgcccaacacagcctatataatagcactagcggcagcccaaaagaacttcactatgaaattgtgcaaggatgctagtgatatcctgcagacgggcaccatccagggagatagggtggcagatgtaattgaaagttccataggagatagcgtgagcagagccctcactcacgctctaccagcacccacaggccagaacacacaggtgagcagtcatcgactggatacaggcaaggttccagcactccaagctgctgaaattggagcatcatcaaatgctagtgacgagagcatgattgagacacgctgtgttcttaactcgcacagtacagctgagaccactcttgatagtttcttcagcagggcgggattagttggagagatagatctccctcttaagggcacaactaacccaaatggttatgccaactgggacatagacataacaggttacgcgcaaatgcgtagaaaggtagagctattcacctacatgcgctttgatgcagagttcacttttgttgcgtgcacacccaccggggaagttgtcccacaattgctccaatatatgtttgtgccacctggagcccctaagccagattctagggaatcccttgcatggcaaaccgccactaacccctcagtttttgtcaagctgtcagaccctccagcgcaggtttcagtgccattcatgtcacctgcgagtgcttatcaatggttttatgacggatatcccacattcggagaacacaaacaggagaaagatcttgaatacggggcatgtcctaataacatgatgggcacgttctcagtgcggactgtggggacctccaagtctaagtaccctttagtggttaggatttacatgaggatgaagcacgtcagggcgtggatacctcgcccgatgcgtaaccagaactacctattcaaagccaacccaaattatgctggcaactccattaagccaactggtgccagtcgcacagcgatcaccactctttag

SEQ ID NO: 10 - protein, 862 aa, Enterovirus 71
MGSQVSTQRSGSHENSNSATEGSTINYTTINYYKDSYAATAGKQSLKQDPDKFANPVKDIFTEMAAPLKSPSAEACGYSDRVAQLTIGNSTITTQEAANIIVGYGEWPSYCSDSDATAVDKPTRPDVSVNRFYTLDTKLWEKSSKGWYWKFPDVLTETGVFGQNAQFHYLYRSGFCIHVQCNASKFHQGALLVAVLPEYVIGTVAGGTGTEDTHPPYKQTQPGADGFELQHPYVLDAGIPISQLTVCPHQWINLRTNNCATIIVPYINALPFDSALNHCNFGLLVVPISPLDYDQGATPVIPITITLAPMCSEFAGLRQAVTQGFPTELKPGTNQFLTTDDGVSAPILPNFHPTPCIHIPGEVRNLLELCQVETILEVNNVPTNATSLMERLRFPVSAQAGKGELCAVFRADPGRNGPWQSTLLGQLCGYYTQWSGSLEVTFMFTGSFMATGKMLIAYTPPGGPLPKDRATAMLGTHVIWDFGLQSSVTLVIPWISNTHYRAHARDGVFDYYTTGLVSIWYQTNYVVPIGAPNTAYIIALAAAQKNFTMKLCKDASDILQTGTIQGDRVADVIESSIGDSVSRALTHALPAPTGQNTQVSSHRLDTGKVPALQAAEIGASSNASDESMIETRCVLNSHSTAETTLDSFFSRAGLVGEIDLPLKGTTNPNGYANWDIDITGYAQMRRKVELFTYMRFDAEFTFVACTPTGEVVPQLLQYMFVPPGAPKPDSRESLAWQTATNPSVFVKLSDPPAQVSVPFMSPASAYQWFYDGYPTFGEHKQEKDLEYGACPNNMMGTFSVRTVGTSKSKYPLVVRIYMRMKHVRAWIPRPMRNQNYLFKANPNYAGNSIKPTGASRTAITTL

SEQ ID NO: 11 - DNA, 2589 bp, Artificial sequence
  Synthetic
atgggatcccaggtgagtacacagcgctccggatcccacgaaaatagcaactccgcaacagagggatctactataaactatacgaccatcaactactataaggactcatacgccgcaacagcaggtaaacaatccttgaagcaggatcctgacaagttcgcgaatcctgttaaggatatattcaccgaaatggcggctccgctgaagtcgccctccgctgaagcttgcggttatagtgatcgtgtagcgcaattgaccattggcaactctaccatcacaacacaggaagccgcaaacatcatcgtgggatacggagagtggccctcgtattgctctgactctgacgctactgcggttgacaagccaactcgtccagacgtttcggtgaacaggttctacaccttggataccaagctctgggagaagagcagcaaaggttggtactggaagtttcctgatgtcctgacagaaactggtgttttcggacagaacgcccagtttcactacctgtatcgttccggcttttgtatccatgtgcagtgcaacgcatcaaagttccaccagggagctcttttggtggccgtattgcccgagtacgtgatcggtaccgttgcaggcggtacaggaaccgaggacacgcacccgccctacaaacaaacgcaaccaggagcggacggtttcgaacttcaacacccctacgttctcgatgctggcattcctatctcacagctgaccgtttgcccacaccaatggatcaacctccgcactaacaactgcgctactattattgtcccttacattaacgcactcccttttgacagtgccctcaaccactgtaacttcggattgcttgtggtccctatatcccccctggactacgaccagggtgcaactccagtgatccctattacgatcaccctcgccccgatgtgttctgagtttgcaggccttcgccaggcggtgacgcagggcttccctacggagttgaagcccggcaccaaccagttccttacgacggacgacggtgtttctgcacccattcttccaaactttcatcccactccatgcatccatatacctggagaagttcgtaacttgctggagctgtgccaggttgaaacgatcctcgaggtcaataacgtcccaactaacgccaccagcttgatggaaagactcagattcccagtgagcgcccaggcgggaaagggagagctgtgcgctgtgtttcgcgctgaccctggacgcaacggtccatggcaaagcacgctcttgggtcagttgtgcggttactatactcaatggtcaggatctctggaagtcactttcatgttcactggcagtttcatggcaactggtaaaatgctgattgcttacacacccccaggcggacctctgccaaaggaccgtgctacggccatgctgggcactcacgtgatttgggatttcggtctccaatcatctgttacgttggtgattccttggatctccaacacgcactatcgcgcccatgcacgcgacggtgtgtttgattattatacgaccggcctggtttcaatctggtaccagaccaactatgttgttccgataggcgccccaaatacggcgtatatcattgccctggccgctgctcaaaagaatttcaccatgaagctgtgcaaggatgcgagcgacatcctgcagaccggtactatccagggagacagggtggctgacgtaatcgagagttctataggtgactcagtttcacgcgctctcacgcacgcgcttcctgctcctaccggccaaaacacacaagtgagctctcataggctggacaccggcaaggtccccgctctgcaggctgcagagatcggcgcctcttcgaacgcatcggatgaatctatgatcgaaacacgttgcgtgcttaactcgcactcaacagcagagactacgctcgactccttcttctcgagggcgggtttggttggagagattgacctcccattgaagggtacgacaaaccccaacggctacgcgaactgggatattgacataaccggttacgcccaaatgagaaggaaagtcgagttgttcacatacatgaggtttgatgctgagtttactttcgtggcttgcacgcccaccggtgaagtcgtgccacagctcctccaatatatgtttgtcccgcccggtgccccgaagcccgattcccgcgaatccctggcttggcaaaccgctacgaacccctcggtctttgtgaaactctccgaccctcctgcgcaagtgtcagtgccttttatgagtcctgccagtgcttatcagtggttctacgatggttacccgactttcggcgagcacaagcaagagaaagatctcgaatacggagcttgtccaaacaacatgatgggtacattctccgtaaggaccgtgggaacgtcgaagtcgaagtatcctcttgtcgtccgcatctatatgagaatgaaacacgttcgcgcttggattccgcgtcccatgcgtaatcaaaattacctgttcaaagccaaccccaactacgctggtaactcaataaagcccacaggtgcaagtcgcaccgcaattactaccttgtag

(19) United States
(12) Patent Application Publication
(10) Pub. No.: US 2015/0044257 A1
(43) Pub. Date: Feb. 12, 2015

(54) BACULOVIRUS-BASED ENTEROVIRUS 71 VLP AS A VACCINE

(71) Applicant: Fred Hutchinson Cancer Research Center, Seattle, WA (US)

(72) Inventors: Denise A. Galloway, Mercer Island, WA (US); Joerg Enssle, Seattle, WA (US)

(73) Assignee: Fred Hutchinson Cancer Research Center, Seattle, WA (US)

(21) Appl. No.: 14/387,159

(22) PCT Filed: Mar. 22, 2013
(86) PCT No.: PCT/US13/33543

§ 371 Date: Sep. 22, 2014

Related U.S. Application Data
(60) Provisional application No. 61/615,175, filed on Mar. 23, 2012.

(51) Int. Cl. A61K 39/125

(52) U.S. Cl. CPC A61K 39/125
USPC 424/216.1

(57) ABSTRACT

An optimized baculovirus/insect cell-mediated system is provided for the production of enterovirus 71 virus-like particles to produce a vaccine against recent EV71 virus outbreaks. Co-expression of the viral capsid polyprotein P1 ORF derived from a fatal case in the Fuyang province of the People's Republic of China plus the 3 CD protease of EV71 prototype strain BrCr resulted in the formation of VLPs. The yields were increased by co-expression of both P1 and 3CD in separate transgene cassettes arranged in opposite orientation in a bicistronic baculovirus vector and by inserting the translational enhancing signal L21 in front of the capsid protein open reading frame. Faster transgene processing was achieved by using insect Sf21 cells instead of Sf9 cells.

### BACKGROUND

Enterovirus 71 (EV71) is an emerging virus with a severe impact on human health. It is an etiological agent responsible for hand-foot-and-mouth disease (HFMD) in young children and infants. EV71 is a picornavirus (picornaviridae), a virus family that is divided into six genera, among which are the human enteroviruses, rhinoviruses, parechoviruses, aphthoviruses, cardioviruses, and hepatoviruses. The genus Enterovirus includes the human virus species of polioviruses and human enterovirus groups A to D. Human enterovirus A consists of 12 serotypes, including coxsackievirus A2, Al6 and enterovirus 71.

Enteroviruses cause a wide spectrum of clinical syndromes ranging from mild fever to respiratory infections, meningitis, encephalitis, paralytic poliomyelitis and myocarditis. Life-threatening enteroviral infections may occur, especially in high-risk individuals such as immunocompromised patients, infants and young children. Reminiscent of experiences with poliovirus, EV71 infection may also have severe neurologic adverse effects (Ooi et al., Lancet Neurol. 9.11 (2010): 1097-105; Solomon et al., Lancet Infect. Dis. 10.11 (2010): 778-90). These include aseptic meningitis, brain stem encephalitis, and death.

Enteroviruses are single-stranded RNA viruses. Their genomes comprise approximately 7500 nts. The genomic organization and replication of EV71 follows the enterovirus prototype genome organization (Kirkegaard, Curr. Opin. Genet. Dev. 2.1 (1992): 64-70): a single open reading frame (ORF) encoding a polyprotein is expressed and subsequently cleaved. The P1 region of the polyprotein gene encodes the four structural virus capsid subunit proteins VP4, VP2, VP3 and VP1. Cleavage of the P1 polyprotein into VP0 (VP4+VP2), VP3 and VP1 is mediated by the viral 3CD protease, the 3CD part of the viral polyprotein, at the plasma membrane.

The EV71 prototype strain BrCr was first described in 1973 (Schmidt et al., J. Infect. Dis. 129.3 (1974): 304-09). Current outbreaks of the emerging subgenotype C4 mainly affect the population in the People's Republic of China (Tan et al., PLoS. One. 6.9 (2011): e25662 (Electronic submission); Zhang et al., J. Clin. Virol. 44.4 (2009): 262-67; Zhang et al., Virol. J. 7 (2010): 94 (Electronic submission); Zhang et al., PLoS. One. 6.11 (2011): e27895 (Electronic submission)) with children under seven years of age being the main susceptible population (Yu et al., Jpn. J. Infect. Dis. 64.6 (2011): 528-32). Currently, there is no curative treatment available for EV71 infection (Thibaut et al., Biochem. Pharmacol. 83.2 (2012): 185-92; Liang et al., Vaccine 29.52 (2011): 9668-74).

Prophylactic vaccines against EV71 are being developed, although these attempts are either directed against formerly EV71 subgenotypes like the EV71 neu strain (Lin et al., Vaccine 20.19-20 (2002): 2485-93; Chung et al., World J. Gastroenterol. 12.6 (2006): 921-27), or are based on inactivated virus (Riedmann, Hum. Vaccin. 7.8 (2011): 802-05; Liang et al., Vaccine 29.52 (2011):9668-74; Chang et al., Vaccine 30.4 (2012): 703-11) with potential risk factors as occurs in poliovirus vaccines (Arita and Francis, Vaccine 29.48 (2011): 8827-34). Safer approaches are needed for vaccine production that are independent from any infectious virus, such as heterologous viral subunit transgene expression systems using, for example, bacteria, yeast, plants or insect baculoviruses (BVs).

One potential approach for vaccine development is the production of virus-like particles (VLPs) using the BV expression system (van Oers, Adv. Virus Res. 68 (2006) 193-253; Kost et al., Nat. Biotechnol. 23.5 (2005) 567-75). Due to the huge coding capacity of BVs large recombinant transgenic insertions can be accomplished. Using one BV containing all heterologous genes of choice greatly simplifies virus handling and has been shown to result in up to a 30-fold higher heterologous protein expression (Berger et al., Nat. Biotechnol. 22.12 (2004): 1583-87), notably in BV-mediated VLP expression systems (Roy et al., Gene 190.1 (1997): 119-29; Bertolotti-Ciarlet et al., Vaccine 21.25-26 (2003): 3885-900).

VLP formation has been accomplished in a BV system with a full-length poliovirus open reading frame (ORF) (Urakawa et al., J. Gen. Virol. 70 (Pt 6) (1989): 1453-63). To produce VLPs of EV71, two components—P1 plus 3CD—have been used together for the EV71 neu strain (Hu et al., Biotechnol. Lett. 25.12 (2003): 919-25; Chung et al., (2006), supra; Chung et al., Vaccine 28.43 (2010): 6951-57) in the insect cell/BV system. Chung et al., ibid, reported a bicistronic BV system for optimal EV71 VLP yield where the P1 gene under the control of the polyhedrin promoter (PHpr), and 3CD under the control of the CMV promoter, are located in a head-to-head promoter configuration. A significant need remains, however, for an efficient system to generate high yields of VLPs of EV71 to develop a protective vaccine, particularly against recent EV71 outbreaks in Asia.

Insect cell lines are used as a culture system for the production of vaccines used in human and veterinary medicine. Many recombinant proteins have been expressed in insect cells that are immunogenically, antigenically, and functionally similar to the native proteins. The desired product is an expressed protein that is produced in large amounts and that is as similar to the natural protein as possible, including necessary post-translation processing and modification. Among the post-translational processing steps that have been shown to occur in insect cells are fatty acid acylation, phosphorylation, and glycosylation (Luckow, V. A. 1995. In: Baculoviruses Expression Systems and Biopesticides, Shuler et al., Eds. Wiley-Liss, New York, N.Y., pages 51-90). Most proteins recovered from insect cell cultures, however, have a lower molecular weight than the native protein because of incomplete post-translational modification. Yields of protein from baculovirus expression vectors in insect cell cultures are reported to be many times higher than those from mammalian cells. Differences in yields of expressed gene products from engineered baculoviruses among cell lines have been reported. Hink and co-workers (Hink et al., Biotechnol Prog. 7 (1991):9-14), compared the expression of three recombinant proteins in twenty-three different cell lines. For each protein, the yield varied among the cell lines and no single cell line produced the highest yields for all three proteins.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect the invention provides a vaccine composition of virus-like particles (VLPs) of human enterovirus 71 (EV71) strain EU703812 for preventing human EV71 infection or inhibiting development of EV71 disease manifestations. Such a vaccine may comprise capsid proteins PV0, VP4, VP2, VP3 and VP1, and preferably will be free of infectious genomic EV71 RNA, and substantially free of any RNA. The vaccine of the invention is preferably available in a concentration of 20 to 100 μg per human dose, together with an adjuvant, such as an aluminum salt. The vaccine may be contained in a vessel such as a vial, capsule, syringe, etc. that is suitable for containing a 500 μl to 1.0 ml human vaccine dose.

In yet additional embodiments the invention provides a method for producing a vaccine against EV71 infection or disease, by infecting Spodoptera frugiperda (Sf) cells with a recombinant baculovirus comprising a P1 gene of EV71 strain EU703812 and a 3CD protease gene of prototype EV71 strain BrCr-Tr under control of a CMV promoter in a bicistronic configuration. The infected Sf cells are cultured under conditions which permit expression of the EV71 genes and assembly of EV71 VLPs, which are then harvested and combined with a physiologically acceptable carrier or an adjuvant to produce the vaccine. The P1 gene is preferably placed under control of a polyhedrin promoter, and/or the 3CD protease gene is preferably under control of a CMV promoter. Preferably the Sf cells are Sf21. The resulting vaccine is preferably free of infectious genomic EV71 RNA.

In further aspects the present invention provides a method for producing a vaccine against EV71 by obtaining EV71 VLPs from a culture of Sf cells, such as, e.g., Sf21 cells, infected with a recombinant baculovirus. The recombinant baculovirus comprises a P1 gene of EV71 strain, which in some instances may be EU703812, and a 3CD protease gene, which in some instances may be from prototype EV71 strain BrCr-Tr, under control of a CMV promoter in a bicistronic configuration. Optionally, a translational enhancer L21 is inserted 5′ of the P1 gene. The infected Sf cells are cultured under conditions which permit expression of the EV71 genes and assembly of EV71 VLPs. The EV71 VLPs are combined with an adjuvant to produce the vaccine.

In another embodiment the invention provides a method for inhibiting or preventing EV71 infection or inhibiting the development of EV71 disease, by administering an effective amount of an EV71 vaccine that comprises EV71 VLPs of strain EU703812, together with a pharmaceutically acceptable excipient and optionally an adjuvant. The vaccine is administered in an amount sufficient to generate an immune response in the individual that prevents EV71 infection or disease, and may be given in two consecutive doses consisting of a first dose and a second dose, where, for example, the second dose is administered at least about two to six months after the first dose. The adjuvant may be an aluminum salt, such as aluminum hydroxide. The EV71 VLPs may comprise capsid proteins PV0, VP3 and VP1 or VP4, VP2, VP3 and VP1 and are free of genomic EV71 RNA.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows the genome structures of recombinant EV71-transgenic BVs. A: Monocistronic BVs. Upper left: BV P1, upper right: BV 3CD, lower left: BV L21 P1, lower right: BV P1 codop. B: Bicistronic BVs. Upper left: BV Bi A, lower: BV Bi B, right: BV Bi B L21. Figure legend: T: Transposon recognition sequence, P: Polyhedrin promoter PHpr, P1: EV71 P1 polyprotein ORF, A: poly A signal light gray: SV40, dark gray: BGH), C: CMV promoter, 3CD: EV71 protease 3CD ORF, L: L21 translation enhancer and P1 codop: codon-optimized EV71 polyprotein ORF. For reasons of clarity the Gentamicin resistance cassette used for selection of recombinant bacmids in bacteria present in all constructs between the left transposon recognition sequence and the PHpr as well as the constant parts of the respective 5′ UTRs of the transgene cassettes are not shown. Vector genomes are not drawn to scale.

FIG. 2 shows the electron microscopic detection of EV71 VLPs. A. Sf9 cells were co-infected with BV P1 and BV 3CD. Following purification steps, VLPs of 30 to 40 nm in size were detected using electron microscopic analysis (indicated by a white arrow for one VLP). The particle labeled with a black arrow might be a BV capsid particle or an immature VLP form. Bar size 200 nm. B. Electron microscopic detection of EV71 VLPs in Sf21 cells after infection with BV Bi B. The white arrow indicates VLP aggregates, putatively forming due to the high expression efficiency when using BV Bi B. Bar size 50 nm.

FIG. 3A: Faster processing of EV71 capsid transgenic proteins in Sf21 cells compared to Sf9 cells and higher EV 71 capsid protein yield through the L21 translational enhancer. Equal amounts of protein were analyzed with the monoclonal antibody (Mab) 10F0. Lane 1: cells infected with BV P1, lane 2: cells infected with BV P1 L21. In these two experiments the cells were co-infected with BV 3CD (multiplicity of infection (MOI) 10). Lane 3: cells infected with BV 3CD only (MOI 10), lane 4: uninfected cells. Upper panel: Sf9 cells, lower panel: Sf21 cells. The EV71 P1 and VP0 proteins are indicated. Putative additional intermediate EV71-specific cleavage products are indicated with asterisks.

FIG. 3B: Superior EV71 capsid protein expression levels from bicistronic BVs in insect cells with the transgene cassettes in opposite orientations. Sf21 cells were infected with BV Bi A (lane 1) and BV Bi B (lane 2) at respective MOIs of 10. Three days post infection cell lysates were harvested. After electrophoretic separation of equal amounts of cellular proteins followed by western blot transfer, EV71 capsid protein expression was detected with the Mab 10F0. Lane 3: uninfected cells.

FIG. 3C: Higher EV71 capsid protein expression in Sf21 cells through the L21 leader sequence in the monocistronic BV infection system but not in the bicistronic configuration. Lane 1: Sf21 cells infected with BV P1 (MOI 10), lane 2: Sf21 cells infected with BV P1 L21 (MOI 10), lane 3: Sf21 cells co-infected with BV P1 (MOI 10) and BV 3CD (MOI 10), lane 4: Sf21 cells co-infected with BV P1 L21 (MOI 10) and BV 3CD (MOI 10), lane 5: Sf21 cells co-infected with BV P1 (MOI 10) and BV 3CD (MOI 1), lane 6: SF21 cells co-infected with BV P1 L21 (MOI 10) and BV 3CD (MOI 1), lane 7: Sf21 cells infected with BV Bi B (MOI 10), lane 7: Sf21 cells infected with BV Bi B L21 (MOI 10), lane 9: uninfected cells. The EV71 P1 and VP0 proteins are indicated. Putative additional EV71-specific intermediate cleavage products are indicated with asterisks. C1. Impaired EV71 VP0 accumulation with L21 in 5′ to the P1 ORF in the bicistronic configuration is detected when fewer amounts of protein are analyzed compared to C for the BV Bi B and BV Bi B L21 infection experiments. Lane 1: Uninfected Sf21 cells, lane 2: BV 3CD-infected cells, lane 3: BV Bi B-infected cells, lane 4: BV Bi B L21 infected cells. Equal amounts of protein were loaded in lanes 3 and 4.

FIG. 3D: Sf21 cells were infected with BV P1 (lanes 1 and 4) or two independent BV stocks with codon-optimized P1 ORF (lanes 2, 3, 5 and 6) at an MOI of 10 either in the absence (lanes 1 to 3) or presence of BV 3CD (MOI 10, lanes 4 to 6). Three days post infection cell lysates were harvested. Lane 7: BV 3CD-infected cells, lane 8: uninfected cells.

FIG. 4: EV71 BrCr VP0 processing into VP2 in 293TT cells in the absence of viral proteases and inhibition of processing in two VP0 alanine mutants. 293TT cells were transfected with plasmids expressing the wt VP0 ORF (lane 1) or two VP0 ORF versions with alanine residues instead of the authentic amino acids at the putative VP4-VP2 cleavage position (lanes 2 and 3). Cell lysates were harvested at day 2 post transfection and subjected to Western blot analysis applying Mab 10F0. Lane 4: Lysate from untransfected 293TT cells, lane 5: lysate from EV71 BrCr-infected 293TT cells.

FIG. 5: BV Bi B infection kinetic in insect cells reveals highest EV71 capsid protein yield five days after infection. Cell lysates harvested at different times post infection were Western blot analyzed with Mab 10F0.

FIG. 6: Purity of EV71 VLPs. Sf21 cells were infected with BV Bi B (MOI 10) and harvested three days post infection. The lysate was subjected to separation on a discontinuous sucrose gradient. The opaque band occurring in the lower part of the 30% sucrose fraction was harvested and dialyzed in a Vivaspin 500 column and separated on 12% Tris/Bis gels. Lane 1: 10 ug BSA, lane 2: 1 ug BSA, lane 3: EV71 VLPs. Left part: proteins visualized with Gel Code Blue. Right part: Western Blot with MAb 10F0. Origins of detectable bands are given. Note that in this gel system VP0 and VP1 run as a doublet. The triplet consisting of the bands A, B and C is discussed in the Example section.

### DESCRIPTION OF EMBODIMENTS

The present invention provides a means to produce a safe and effective vaccine to quickly respond to EV71 outbreaks, such as occurred recently in the People's Republic of China and elsewhere in Asia. According to this invention, the EV71 P1 gene derived from a recent fatal case in China, and the 3CD protease from the BrCr-TR prototype strain, are co-expressed by a baculovirus (BV)/insect cell system. The EV71 P1 ORF is placed under control of a promoter, such as, for example, the BV polyhedrin promoter (PHpr) (Smith et al., Mol. Cell Biol. 3.12 (1983): 2156-65; Pennock et al., Mol. Cell Biol. 4.3 (1984): 399-406). The 3CD protease of the prototype strain BrCr-TR (Arita et al., J. Gen. Virol. 86.Pt 5 (2005): 1391-401) is placed under the control of a different promoter, such as, by way of example, the cytomegalovirus (CMV) promoter (Boshart et al., Cell 41.2 (1985): 521-30). Using such a strategy as described herein, VLPs of EV71 capsid proteins are generated. The invention thus provides a highly efficient means to produce VLPs that can be used to formulate a protective vaccine against EV71 outbreaks.

To increase VLP yield, the invention augments the rate of translation from EV71-specific mRNA transcripts in BV-infected insect cells. The translational enhancer sequence L21 is inserted into 5′ of the P1 ORF and leads to a much higher amount of processed P1 subunits. EV71 capsid protein expression was not found to be augmented by using a codon-optimized approach, in contrast to codon-optimization in other BV-systems that led to increased production of the respective heterologous transgene (Angov, Biotechnol. J. 6.6 (2011): 650-59; Zhang et al., Biochem. Biophys. Res. Commun. 227.3 (1996): 707-11). However, the use of Sf21 cells was found to be superior to Sf9 cells, and thus useful in producing EV71 VLP on a large scale.

In the bicistronic systems both the same gene cassettes—albeit with different EV71 ORFs and other slight modifications like the polyadenylation signals used—were either in a sense configuration with the P1 cassette positioned in 5′ to the 3CD cassette, or in antisense orientation with adjacent polyadenylation cassettes. Proper proteolytic P1 processing was observed with both versions, implying functional expression of P1 and 3CD, i.e., the positions and orientations of the transgene cassettes in both constructs allowed BV-mediated transgene production in insect cells. Thus the invention supports that both transgene cassettes can be arranged in the described configuration suggesting at least a partial non-interfering activity of both promoters, and a qualitative superiority in protein expression with the opposite orientations.

Contradictory to picornavirus dogma that VP0 processing into VP2 and VP4 requires viral RNA encapsidation, the VP0 cleavage into VP2 and VP4 may also occur without encapsidation of viral genomic RNA into a preformed capsid, and even if only the VP0 subunit itself is present in a cell. It is likely that the final proteolytic VP0 processing step into VP2 and VP4 observed in lysates of infected insect cells and in 3CD-deficient 293TT cells after expression of VP0 leads to mature VLPs containing processed VP2 and VP4 even in the absence of encapsidated viral genomic RNA. At least a certain percentage of EV71 VLPs would more resemble fully processed infectious viral capsids.

In the present invention, VLPs derived from the supernatant of infected Sf21 cells were not observed with VP2, implying that in particles released from infected insect cells the final maturation VP0 cleavage into VP2 and VP4 would not occur. This indicates a qualitative difference between supernatant-derived VLPs and cell-associated, for which a partial maturation was observed of VP0 into VP2 and VP4. The shape of a fully matured VLP would more closely resemble an infectious viral particle and therefore trigger a protective vaccine-based immune response more efficiently and more specifically to viral capsid domains suggesting a qualitative difference in protective immunity depending on whether the VLP source is either infected insect cells or the cell supernatant.

VLPs and Vaccine Use

As used in herein, “virus-like particles” or “VLPs” refers to virus particles of EV71 that self-assemble into intact virus structures comprised of capsid proteins such as EV71 capsid proteins. VLPs are morphologically and antigenically similar to authentic virions, but do not contain genetic information sufficient to replicate and thus are non-infectious. VLPs are produced in suitable host cells (i.e., insect host cells) wherein upon isolation and further purification under suitable conditions they are purified as intact VLPs. As disclosed herein, “mutation” includes substitutions, transversions, transitions, transpositions, reversions, deletions, insertions, or other events that may have improved desired activity, or a decreased undesirable activity of the gene. Mutation encompasses null mutations in natural virus isolates or in synthesized genes that may change the primary amino acid sequences of the expressed protein but do not affect self-assembly of capsid proteins, and antigenicity or immunogenicity of VLPs or chimeric VLPs. Virus-like particles typically self assemble in the cell and remain intracellular; therefore isolation of these particles requires processes of cell disruption and protein solubilization with the accompanying risks of VLP disruption, proteolysis and contamination of the end product. In some cases infected cells extracellularly express viral capsid proteins that self assemble into VLPs.

The invention thus provides a vaccine comprising VLPs produced according to the methods described herein. The vaccine or medicament is preferably used for protection against and/or treatment of an EV71 related disease. Said vaccine or medicament can be administered to an individual at risk of said disease, preferably in combination with a suitable adjuvant and/or carrier, before an EV71 infection has taken place. Such immunization provides a degree of protection against subsequent infection and disease symptoms. The VLP produced according to the invention may in some instances be administered as a medicament after an EV71 infection. The medicament will at least in part counteract EV71 infection. A medicament comprising a VLP molecule of the invention can of course be combined with one or more other medicaments, such as inflammation inhibitors. In one embodiment several medicaments or vaccine preparations are separately administered to an individual. However, a VLP vaccine or medicament molecule of the invention can also be combined with another pharmaceutically active compound in one pharmaceutical or vaccine preparation.

VLPs of the invention can be made by disassembly and reassembly techniques. For example, as described in McCarthy et al., 1998 J. Virology 72(1):33-41, employing the disassembly and reassembly of recombinant human papilloma virus VLPs purified from insect cells to obtain a homogeneous preparation of VLPs. U.S. Pat. No. 6,245,568 also describes a general disassembly/reassembly process for making HPV VLPs that can be employed in the context of preparing VLPs of the present invention.

VLP formation can be assessed by standard techniques such as, for example, electron microscopy and dynamic laser light scattering.

Optionally the vaccine can also be formulated or co-administered with VLPs of other strains of EV71, or with other, non-EV71 antigens. Suitably these non-EV71 antigens can provide protection against other diseases that typically affect the targeted patient population of infants and young children. For example, the vaccine may provide protection against both EV71 and rotavirus.

In one embodiment the vaccine is provided in a liquid vaccine formulation, although the vaccine can be lyophilized and reconstituted prior to administration.

The vaccine described herein can be formulated to comprise an adjuvant or a mixture of adjuvants, in combination with the VLPs. The VLPs can be used in combination with aluminum, and can be adsorbed or partially adsorbed onto aluminum adjuvant. Other adjuvants which can be used are adjuvants which stimulate a Th1 type response such as lipopolysaccharides, for example a non-toxic derivative of lipid A, such as monophosphoryl lipid A or more particularly 3-O-desacyl-4′-monophoshoryl lipid A (3D-MPL). Suitably the adjuvant is an aluminum salt, preferably in combination with a lipopolysaccharide such as 3D-MPL. Thus, in one embodiment the adjuvant is aluminum hydroxide, or the combination of aluminum hydroxide with 3D-MPL.

When VLPs of the invention are adsorbed onto aluminum containing adjuvants, the VLPs can be adsorbed to the aluminum adjuvant prior to mixing of the VLPs to form the final vaccine product. The vaccine can also comprise aluminum or an aluminum compound.

The vaccine described herein can be administered by any of a variety of routes such as oral, topical, subcutaneous, musosal, intraveneous, intramuscular, intranasal, sublingual, intradermal and via suppository. Oral, intramuscular and intradermal deliveries are preferred.

The EV71 vaccine prepared as described herein can be tested using standard techniques, for example in standard preclinical models, to confirm that the vaccine is sufficiently immunogenic.

For the vaccines described herein, in one embodiment a vaccine is used for the vaccination of children aged from 6 months and older, e.g., such as 6 months to 4 years. However, children above 4 years old can also be vaccinated. Similarly the vaccine can be administered to older age groups such as 8 to 12 years and older or women of childbearing age.

In one embodiment, the vaccine of the present invention is administered in one, two or three doses wherein each dose of the vaccine comprises EV71 VLPs in a concentration of greater than about 10 μg up to about 1 mg, more typically from about 15 to 20 μg up to 500 μg, more often 20 μg up to about 100 or 200 μg per dose. In one embodiment, the vaccine is administered in two or three doses wherein each dose of the vaccine comprises EV71 VLPs in a concentration of greater than 20 μg, for example, 30 μg of VLP, or 40 μg of VLP, or 60 μg of VLP, together with an adjuvant.

Administration of the vaccine can follow any appropriate dosing schedule, e.g., a 2-dose schedule, for example a 0, 1 month schedule, a 0, 2 month schedule, a 0, 3 month schedule, a 0, 4 month schedule, a 0, 5 month schedule or a 0, 6 month schedule. For example the second dose is administered between 2 weeks and 8 months after administration of the first dose, for example between 1 and 6 months after the first dose or between 3 and 8 months after the first dose. Thus the second dose may be administered for example one month or two months or three months or four months or five months or six months after the first dose.

In one embodiment the second dose of vaccine is administered more than two months after the first dose, for example 3 or more months, or 4 or more months, or 5 or more months, or 6 or more months after the first dose, where in each case there can be an upper limit of 8 to 12 months after the first dose. The vaccine, use or method can employ EV71 VLPs, each in an amount greater than 10 to 20 μg per human dose, for example 30 μg per dose or greater than 30 μg per dose, for example 40 μg per dose or 60 μg per dose or 80 μg per dose. The amount of EV71 VLPs per dose can be the same or different.

The term “vaccine” as used herein refers to a composition that comprises an immunogenic component capable of provoking an immune response in an individual, such as a human, wherein the composition may be formulated to optionally contain an adjuvant. A vaccine for EV71 suitably elicits at least a partial protective immune response against infection, or persistent infection, more preferably a complete protective immune response against infection. By the term “human dose” is meant a dose which is in a volume suitable for human use and may be contained in a single dosage unit container, such as a vial, syringe or other suitable vessel. Generally this is a liquid between 0.3 and 1.5 ml in volume. In one embodiment, a human dose is 0.5 ml. In a further embodiment, a human dose is higher than 0.5 ml, for example 0.6, 0.7, 0.8, 0.9 or 1 ml. In a further embodiment, a human dose is between 1 ml and 1.5 ml.

Baculovirus Expression Systems

A preferred expression vector of the invention is a baculovirus vector. For baculovirus vectors and baculovirus DNA, as well as insect cell culture procedures, see, for example, O'Reilly et al., Baculovirus Expression Vectors: A Laboratory Manual, Oxford University Press, New York, 1994, incorporated herein by reference in its entirety. The baculovirus vector construct of the invention preferably contains additional elements, such as an origin of replication, one or more selectable markers allowing amplification in the alternative hosts, such as E. coli and insect cells. Insect host cells include, for example, Lepidopteran cells, and particularly preferred are Spodoptera frugiperda, Bombyx mori, Heliothis virescens, Heliothis zea, Mamestra brassicas, Estigmene acrea or Trichoplusia insect cells. Non-limiting examples of insect cell lines include, for example, Sf21, Sf9, High Five (BT1-TN-5B1-4), BT1-Ea88, Tn-368, mb0507, Tn mg-1, and Tn Ap2, among others.

In certain embodiments, there are provided baculovirus vectors that contain cis-acting control regions effective for expression in a host operatively linked to the polynucleotide to be expressed. Appropriate trans-acting factors are either supplied by the host, supplied by a complementing vector or supplied by the vector itself upon introduction into the host.

Host cells are genetically transformed to incorporate EV71 P1 and 3CD protease polynucleotides and express polypeptides of the present invention. The recombinant vectors containing a polynucleotide of interest are introduced into the host cell by any of a number of appropriate means, including infection (where the vector is an infectious agent, such as a viral or baculovirus genome), transduction, transfection, transformation, electroporation, microprojectile bombardment, lipofection, or combinations thereof. A preferred method of genetic transformation of the host cells, according to the invention described herein, is infection.

The polynucleotides are introduced alone or with other polynucleotides. Such other polynucleotides are introduced independently, co-introduced or introduced joined to the polynucleotides of the invention. Thus, for instance, a polynucleotide (i.e., P1 gene) is transfected into host cells with another, separate polynucleotide (i.e., X2 or fusion X2 genes) using standard techniques for co-transfection and selection. In another embodiment, the polynucleotides encoding P1 capsid protein and the polynucleotides encoding X2 protein or an L2 fusion protein are present on two mutually compatible baculovirus expression vectors which are each under the control of their own promoter.

The present application claims the benefit of U.S. application 61/615,175, filed Mar. 23, 2012, which is incorporated by reference herein in its entirety.

### EXAMPLES

Methods

Cloning Procedures

All PCR-based cloning procedures were carried out using Platinum Taq polymerase (Invitrogen, Carlsbad, Calif.). All primers were purchased from Integrated DNA Technologies Inc. (IDT, Coralville, Iowa). Restriction enzymes and DNA modifying enzymes were purchased either from Invitrogen or New England Biolabs (NEB, Ipswich, Mass.), respectively.

The EV71 3CD protease/polymerase ORF was amplified from pEV71 (BrCr-TR) containing the sequence for the prototype EV71 strain BrCr-TR ((Arita et al., J. Gen. Virol. 86.Pt 5 (2005): 1391-1401)) using primers 3CD Start: [SEQ ID NO:1] 5′-GGCGCGGCCGCATGGGGCCCAGCTTAGACTTCGCCTTGTCT-3′ and i-3CD Stop: [SEQ ID NO:2] 5′-CGCCTCGAGTTAAAATAACTCCAGCCAATTTCTTCTC AAGT-3′, respectively, for 40 cycles at 30″ at 95° C., 30″ at 50° C. and 5′ at 72° C. The 1961 bp 3CD fragment was digested with NotI and XhoI and inserted into p16mSV40ori L1 (a modified version of plasmid p16L1rLOCUSm) digested with NotI and SalI thus generating p16m SV40ori 3CD. A 2603 bp NotI-SalI DNA fragment from p16m SV40ori P1 was cloned into NotI-XhoI digested pFastBac1 (Invitrogen). A 2174 bp NotI-SphI fragment from p16mSV40ori3CD encoding for the EV71 3CD ORF was inserted into pFastBac1 digested with NotI and SphI leading to pFastBac 3CD.

Two synthetic genes encoding P1—either derived from a recent EV71 isolate from the People's Republic of China's province Fuyang (Zhang et al., Virol. J. 7 (2010): 94 (Electronic submission) (http://www.ncbi.nlm.nih.gov/nuccore/EU703812.1), and the second one encoding the same amino acid sequence but codon-optimized for BV/insect cells (Nakamura et al., Nucleic Acids Res. 28.1 (2000): 292) were purchased from IDT.

The respective nucleotide sequences (set forth below) for the P1 ORF were equipped with [SEQ ID NO:3] 5′-GCGGCCGCTCTAGACC-3′ as 5′-untranslated region (UTR) and [SEQ ID NO:4] 5′-TCGACAAGCTT-3′ as 3′-UTR, respectively. The cloned authentic P1 ORF was inserted as a NotI-SalI fragment into NotI-XhoI digested pFastBac 1 leading to pP1 NS. The codon-optimized P1 ORF was cloned in the same way leading to pP1 codop.

For the construction of a bicistronic BV with EV71 P1 controlled by PHpr and the 3CD protease under the control of the CMV promoter a 2710 bp PCR fragment encoding the CMV promoter plus EV71 3CD protease and the bovine growth hormone (BGH) poly-adenylation signal (Hampson et al., Proc. Natl. Acad. Sci. U.S.A 84.9 (1987): 2673-77) was amplified from p16mSV40ori3CD with primers AvrII sense [SEQ ID NO:5] (5′-GGCCTAGGGTATTAGTCATCGCTATTACCA-3′) and AvrII anti [SEQ ID NO:6] (5′-GGCCTAGGTCCCCAGCATGCCTGCTATTGT-3′). After digestion with AvrII the PCR fragment was inserted into AvrII-digested pP1 NS. Because of the cloning strategy two versions with different 3CD transgene insert orientations were obtained. Both recombinant variants, termed pBi A and pBi B, respectively, were used for further analysis. For positioning the L21 translational enhancer sequence (Sano et al., FEBS Lett. 532.1-2 (2002): 143-46) upstream of the P1 ORF, a 996 bp PCR fragment was generated using primers PCR L21s [SEQ ID NO:7] (5′-CTCTAGAAGCTTCCTAAAAAACCGCCACCATGGGTTCGCAAGTGTCTA-3′) and i-PV0 RF [SEQ ID NO:8] (5′-GCGTGACTGCCTGCCTAAGACC-3′) with pP1 NS as template. After digestion with XbaI and PmlI, the PCR fragment encoding for the optimized translation initiation sequence upstream of the P1 ORF was inserted into XbaI-PmlI-digested pP1NS leading to pP1 L21. A bicistronic BV expression plasmid with the CMV-promoted 3CD ORF and the PHpr-driven Chinese P1 ORF with the improved translation initiation sequence L21 positioned in 5′ to the P1 ORF was cloned by inserting a 3319 bp BsrGI-KpnI fragment from pP1 L21 into the BsrGI-KpnI digested pBi B. The sequence integrity of all PCR-amplified cloning fragments was verified in the final basic plasmid clones.

Recombinant EV71 Subunit Bacmid Cloning

Production of recombinant bacmids containing EV71 subunits P1 from the

Chinese subgenotype and 3CD (Br-Cr-TR) was carried out using the BAC-to-BAC expression system (Invitrogen) according to the manufacturer's instructions using plasmids pP1 NS, pFastBac 3CD, pP1 L21, pP1 codop, pBi A, pBi B and pBi B L21, respectively, to generate the bacmids BAC P1 NS, BAC 3CD, BAC P1 L21, BAC P1 codop, BAC Bi A, BAC Bi B and BAC Bi B L21. Bacmids were purified using a commercially available nucleic acid isolation kit (Invitrogen). A PCR-based colony screening was carried out to identify EV71 subunit-positive bacmids with combinations of primers Ml3Forward or Ml3Reverse and EV71 gene-specific primers, respectively.

Cell Culture, Transfections and BV Titrations

Spodoptera frugiperda (Sf) 9 insect cells (Vaughn et al., In Vitro 13.4 (1977): 213-17), Sf21 cells (Vaughn et al., id.) and the SF Easy Titer cell line ((Hopkins et al., Biotechniques 47.3 (2009): 785-88)) were cultivated in SF-900II SFM medium (Invitrogen) with 10% FBS (Gemini Bio-Products, West Sacramento, Calif.) and antibiotics at 30° C. in a humidified atmosphere. Cellfectin II (Invitrogen) was used to transfect recombinant bacmid DNA into insect cells according to the manufacturer's instructions. Recombinant EV71-transgenic BV stocks were prepared from infected cell supernatants by filtrating through 0.22 μm filters and titrated using the SF Easy Titer cell line according to the authors' instruction.

Protein Analysis

Infected or control (Mock) infected insect cells were harvested from tissue culture dishes and pelleted (5 min at 8000 rpm). After resuspending in PBS they were subjected to one freeze/thaw cycle (liquid N₂). Insoluble debris was pelleted from the protein lysates in an Eppendorf centrifuge at 8000 rpm for 5 minutes at room temperature. Protein concentrations of lysates were measured with a Nanodrop analyzer (Fisher Scientific, Rockford, Ill.).

Lysates were electrophoretically separated on 12% Tris/Bis protein gels (Invitrogen) and transferred to nitrocellulose membranes (Micron Separations, Inc, Westborough, Mass.). For comparative analyses and comparing loaded protein amounts, membranes were stained with Ponceau red (Invitrogen) to corroborate the transfer of equal amounts of whole protein. EV71 protein expression was detected using the anti-EV71 monoclonal antibody (Mab) 10F0 (Abcam, San Fransisco, Calif.) at 1:10000 in PBS with 5% dry milk powder at 4° C. overnight. After washing with 0.5% Tween in PBS, specifically bound antibody was detected using a horse radish peroxidase (HRP)-labeled goat anti-mouse (1:20000) antibody (Jackson ImmunoResearch Laboratories, West Grove, Pa.) and the Lumi Light detection kit (F. Hoffmann-LaRoche Ltd., Basel, Switzerland) with KODAK X-OMAT Blue films. Occasionally, signals on western blot films were analyzed with the ImageJ software program (http://imagej.nih.gov/ij/).

VLP Preparation by Ultracentrifugation and Electron Microscopic Detection

EV71 VLPs were purified from the lysate of infected cells by physico-chemical steps as follows: The cells were harvested from tissue culture dishes and pelleted (1500 rpm, 5 min). The supernatant was removed, and the cell pellet was resuspended in PBS. After one freeze/thaw cycle in liquid N₂ insoluble debris was pelleted (8000 rpm, 5 min, Eppendorf centrifuge). The supernatant was loaded on a discontinuous sucrose gradient (15%, 30% and 65%) and fractionated by ultracentrifugation (SW55TI rotor, 15000 rpm, 3 hrs, 4° C.). For each sucrose concentration two fractions (upper and lower) were harvested. Aliquots of each fraction were subjected to Western Blot analysis using Mab 10F0. The fraction containing the highest detectable amount of VP0 was concentrated and dialyzed using Vivaspin 500 columns (MW cutoff 100 kDa, Sartorius Stedim Inc., Bohemia, N.Y.). Aliquots were fixed with 3% uranyl acetate followed by negative staining EV71 VLPs were detected by electron microscopy using a JEOL 1230 transmission electron microscope. Alternatively, aliquots were separated by SDS-PAGE and stained with GelCode blue reagent (a coomassie G-250 stain; Pierce Biotechnology, Rockford, Ill.) with BSA standards (NEB) or subjected to Western Blot analysis using Mab 10F0.

Results

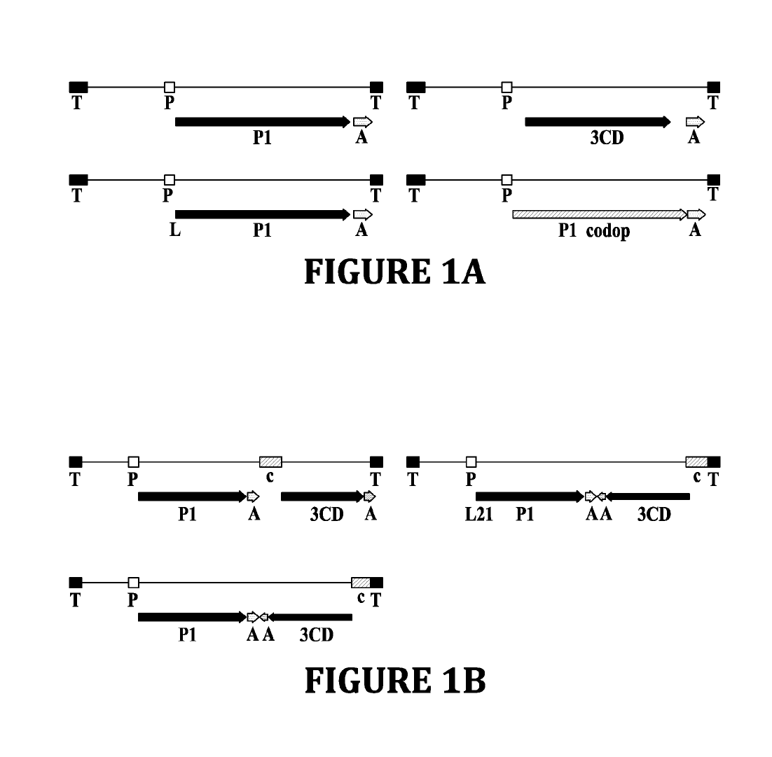

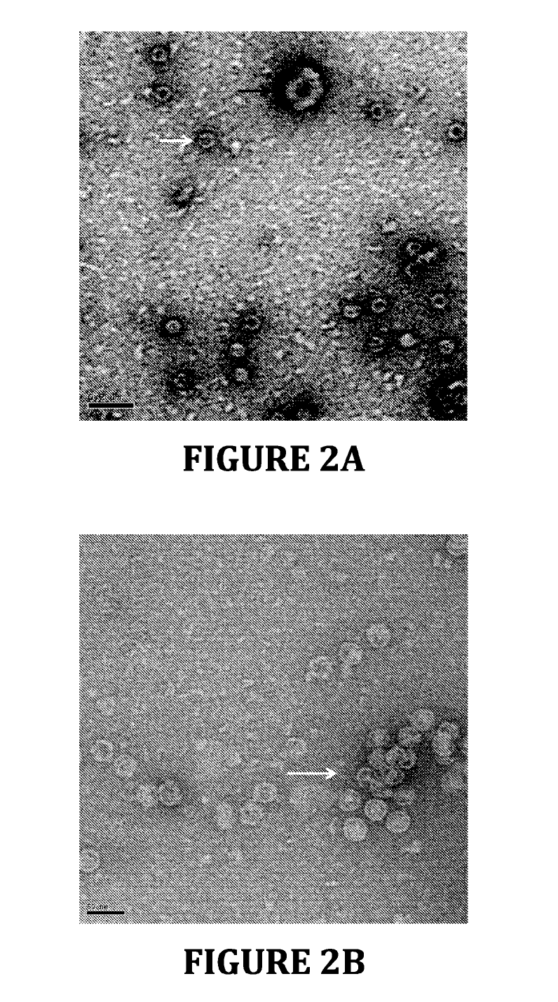

The viral 3CD protease ORF derived from the EV71 prototype strain BrCr was expressed using PHpr (FIG. 1, BV 3CD). Sf9 cells were co-infected with BV P1 and BV 3CD, and cell lysates were analyzed with a commercially available monoclonal antibody (see above) recognizing the EV71 capsid protein P1 and its subunits VP0 and VP2. Using this antibody, VP0 was detected in the lysate of infected cells, indicating that the prototype BrCr strain 3CD protease recognizes P1 as a substrate. The lysate of these cells was further subjected to ultracentrifugation and further VLP purification steps. EV71 VLPs were then detected using electron microscopic techniques (FIG. 2A).

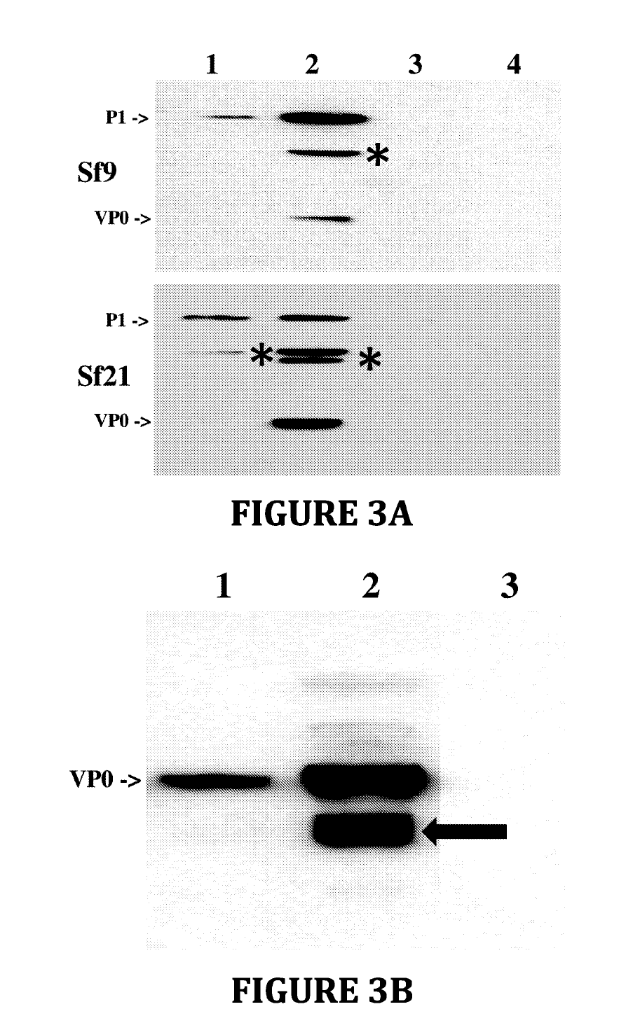

Next, production of EV71 capsid subunit proteins was optimized in the insect cell/BV system. First, to increase the amount of P1 protein as a substrate for EV71 VLPs the translational enhancer sequence L21 was inserted into the P1 transgene cassette (FIG. 1, BV P1 L21). The impact of L21 on P1 synthesis was determined by measuring the VP0 subunit levels in infected cells in a monocistronic co-infection system with the P1 variants and 3CD on separate BVs, both EV71 transgenes being controlled by PHpr. Sf9 and Sf21 cells were infected with BV P1 EU and BV P1 L21 respectively, at an MOI of 10 and co-infected with BV 3CD at an MOI of 10. Three days post infection the cells were harvested and analysed for EV71 capsid subunit protein expression (FIG. 3A). The amount of L21-dependent VP0 synthesis increased dramatically (compare lanes 1 (authentic P1 expression) vs. lanes 2 (L21-driven P1 expression)). This finding could be observed in Sf9 and in Sf21 cells, respectively (compare FIG. 3B, upper panel (Sf9 cells) vs. lower panel (Sf21 cells). Additional bands of ca. 62 and 55 kDa were detectable with Mab 10F0 (FIG. 3A, respective lanes land 2, labelled with asterisks).

Next, the quantitative VP0 expression using the bicistronic BVs Bi A and BV Bi B with P1 expression driven by the PHpr and 3CD expression controlled by the CMV promoter (FIG. 1, BV Bi A and BV Bi B) was compared. Sf21 cells were infected with viruses at MOIs of 10 and harvested two days post infection. Interestingly, much higher VP0 expression was found with the Bi B configuration with the transgene cassettes in opposite transcriptional orientation (FIG. 3B, lane 2) whereas from the Bi A configuration with the transgene cassettes in a sense transcriptional orientation the amount of EV71 capsid subunit proteins was lower by approximately a factor of 7 (FIG. 3B, lane 1).

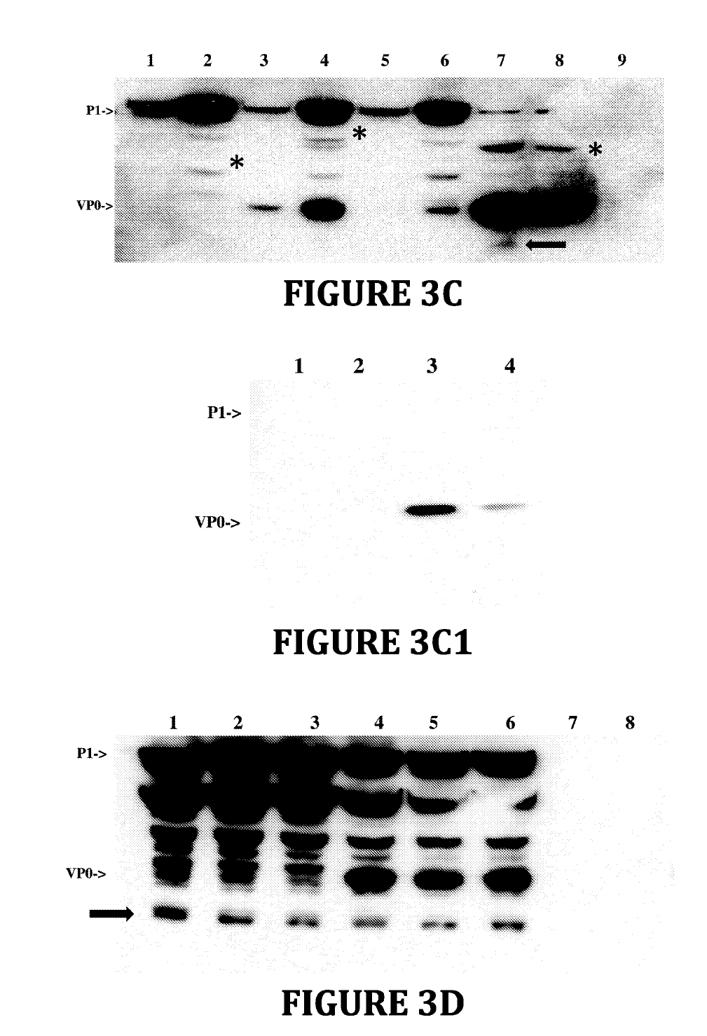

Furthermore, apart from the dominant 35.2 kDa VP0 capsid protein subunit also observed was an additional ca. 28 kDa EV71-specific capsid protein band apparently consisting of two almost equal-sized proteins in BV Bi B-infected cells (see FIG. 3A, lane 2, labeled with a red asterisk), comparable in size to EV71 VP2. These proteins were also detectable in lysates of BV Bi A-infected cells if the western blot was subjected to a longer EV71 protein expression detection time. Such a VP2-like band was also observed when the EV71 BrCr VP0 ORF was transiently expressed in 3CD-deficient 293TT cells (FIG. 4, lane 1). Of note, these additional VP2 capsid proteins did not appear after transient VP0 expression when the putative VP0 cleavage site was mutated (FIG. 4, lanes 2 and 3).

The L21 translation enhancing sequence was then used in the bicistronic BV with P1 and 3CD cassettes in opposite orientations and the bacmid Bi B L21 was constructed with both cassettes opposed to each other but with the L21 leader sequence in 5′ to the P1 ORF. However, when directly compared, EV71 capsid protein expression from BVs derived from this construct indicated no further increase in VP0 capsid protein synthesis (FIG. 3C, lane 8) compared to the L21-deficient bicistronic version (FIG. 3C, lane 7). Rather, VP0 accumulation seemed to be decreased when fewer equal amounts of cell lysates were analyzed to counteract saturation effects during western blot analysis (FIG. 3D, compare lanes 3 (BV Bi B) and 4 (BV Bi B L21)). However increased VP0 production was observed in the bicistronic configuration (FIG. 3C, lane 7) compared to the monocistronic BV infection approach even with L21 being present in front of the P1 ORF (FIG. 3C, lane 4). In this approach, 3CD expression was accomplished by co-infecting the cells with BV 3CD at an MOI of 10. Again, additional EV71-specific capsid proteins of ca. 6 and 55 kDa plus a 45 kDa band (labelled with asterisks) were observed. Also detected was increased VP0 accumulation when MOIs of 10 were used for BV 3CD instead of an MOI of 1 (compare FIG. 3C, lane 3 (BV 3CD MOI 10) versus lane 5 (BV 3CD MOI 1) for the authentic P1, and lane 4 (BV 3CD MOI 10) versus lane 6 (BV 3CD MOI 1) for L21-P1).

Increased P1 synthesis was attempted from an ORF having been codon-optimized for BV/insect cell expression (FIG. 1, BV P1 codop). However when the impact of using a codon-optimized version of the P1 ORF in BV-infected insect cells was investigated, a decisive increase in P1 steady state expression levels in cells infected with BVs with the authentic P1 ORF (FIG. 3 D, lane 1) was not observed when compared cells having been infected with two independently generated BV stocks with a codon-optimized P1 ORF (FIG. 3D, lanes 2 and 3). Nor was increased VP0 accumulation observed when using the codon-optimized P1 ORF when the cells were co-infected with BV 3CD (FIG. 3D, lane 4: authentic P1 versus lanes 5 and 6: codon-optimized P1 ORF). Therefore, and because the use of higher MOIs of BV 3CD during co-infection seemed to be unreasonable for logistic reasons to achieve higher yields of EV 71 capsid subunit protein synthesis, VLP production with the bicistronic BV Bi B with the highest observed VP0 yields was pursued. Electron microscopic morphology and shape of EV71 VLPs obtained after BV Bi B-mediated production was indistinguishable from VLPs obtained with the monocistronic prototype BVs yet the occurrence of VLP aggregates was most likely due to the higher production efficiency obtained when using BV Bi B (FIG. 2B).

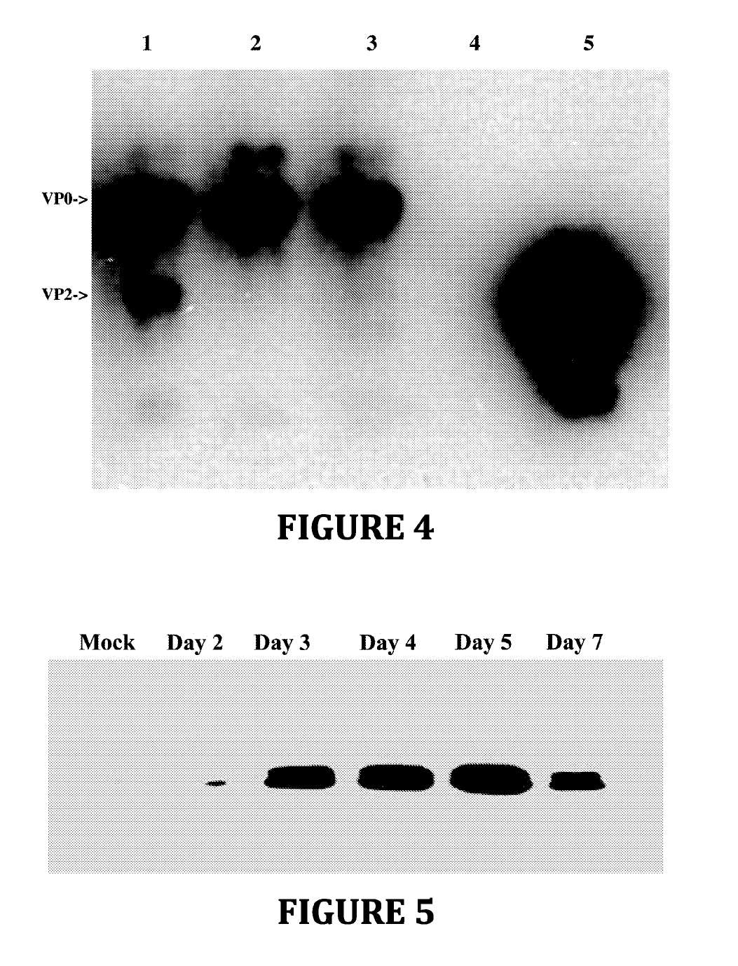

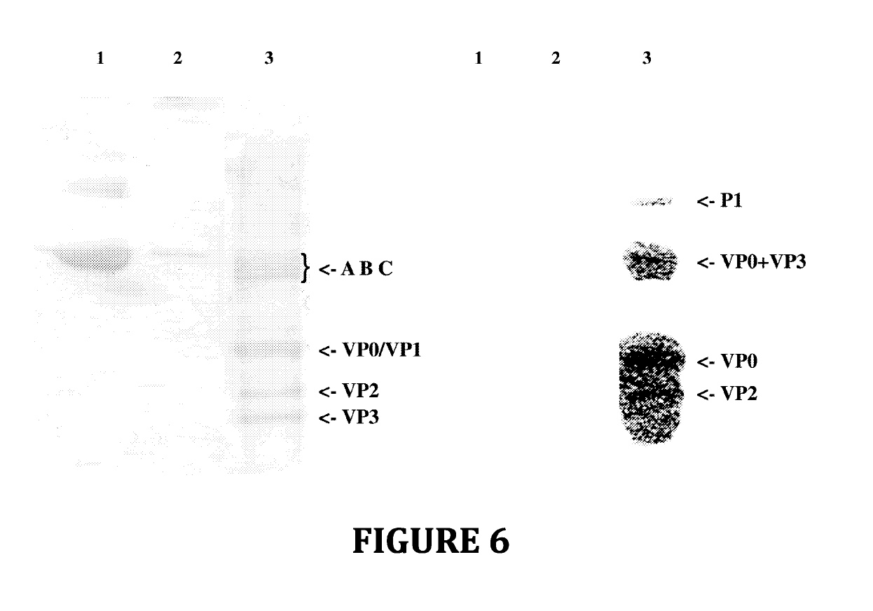

For large scale preparation the time of the highest VLP yield was sought more precisely. For that Sf21 cells were infected with BV Bi B (MOI 10) and cells harvested over six days. When analyzing equal amounts of protein from infected cells the highest amount of EV71 VP0 capsid subunit was detected at day five post infection (FIG. 5). VLPs were then prepared from Sf21 cells using preparative ultracentrifugation (see Methods, supra) and analyzed for yield and purity (FIG. 6). In addition to the capsid subunits PV0, VP3 and VP1 the presence of VP2-like proteins was also observed in the VLP fraction (FIG. 6, left and right parts) plus additional bands reminiscent of the proteins having been detected by Western Blot earlier in the cell lysates. Additionally, P1 and VP2-like proteins could be detected using Mab 10F0 (FIG. 6, right part). Furthermore, a protein band triplet was detected after protein gel staining (FIG. 6, left part, “A B C”) with one of these three bands most likely being unprocessed PV0-VP3 proteins (FIG. 6, right part, “VP0+VP3”).

Discussion

Recombinant BVs were constructed expressing the P1 ORF isolated from a fatal case of EV71 infection during a recent outbreak in the People's Republic of China. The P1 ORF was placed under the control of the PHpr (Smith et al., Mol. Cell Biol. 3.12 (1983): 2156-65; Pennock et al., Mol. Cell Biol. 4.3 (1984): 399-406), and the 3CD protease of BrCr-TR (Arita et al., J. Gen. Virol. 86.Pt 5 (2005): 1391-401) was under the control of the CMV promoter (Boshart et al., Cell 41.2 (1985): 521-30). Using this strategy, VLPs were generated. Then efforts were directed toward further increasing

VLP yield by augmenting the rate of translation from EV71-specific mRNA transcripts in BV-infected insect cells.

Following a hypothesis that high levels of P1 ORF protein would lead to high amounts of VLPs, the enhancer sequence L21 was inserted 5′ of the ORF. It was also investigated whether EV71 capsid protein expression could be improved by codon-optimization, which has in other BV-systems led to increased production of the respective heterologous transgene (Angov, Biotechnol. J. 6.6 (2011): 650-59; Zhang et al., Biochem. Biophys. Res. Commun. 227.3 (1996): 707-11). The BV with the highest observed VP0 capsid subunit yields in small-scale experiments were used to produce EV71 VLPs.

After co-expression of EV71 recombinant BVs P1 and 3CD, VP0 could be detected in protein lysates from infected Sf9 cells implying that the EV71 BrCr prototype 3CD protease recognizes P1 from the Chinese isolate as a substrate. That is not insubstantial because although the amino acids at the boundaries between VP0 and VP3 at the first cleavage site and VP3 and VP1 at the second cleavage site are both conserved Gln-Gly residues in the Chinese P1 protein, the substrate for picornavirus 3CD protease (Kean et al., J. Gen. Virol. 71 (Pt 11) (1990): 2553-63), the amino-terminal residue next to the PV0-VP3 Gln-Gly junction is different in the Chinese strain (Thr) compared to BrCr-TR strain (Ala). Also, the third residue next to the VP3-VP1 Gln-Gly junction in the VP3 protein is a Gly in the Chinese P1 and an Ala in the BrCr-TR strain. Such substrate changes could effect or even abolish the cleavage of the Chinese P1 into PV0, VP3 and VP1 by the prototype BrCr-TR strain 3CD protease.

Electron microscopic analysis clearly showed the presence of EV71 VLPs of 30 to 40 nm in size and icosahedral shape in EV71-transgenic BV-infected insect cells. Encouraged by this finding, efforts towards a BV-based EV71 vaccine were directed to optimize EV71 transgene expression by genetic approaches. Constructs were made to express the P1 variant from recent EV71 outbreaks in China. In the bicistronic systems both the same gene cassettes were either in a sense configuration with the P1 cassette positioned in 5′ to the 3CD cassette, or in antisense orientation with adjacent polyadenylation cassettes. Nevertheless, proper proteolytic P1 processing was observed with both versions, implying functional expression of P1 and 3CD, i.e., the positions and orientations of the transgene cassettes in both constructs allowed BV-mediated transgene production in insect cells. The complex genome organization of BV provides examples of comparable viral gene arrangements (Ayres et al., Virology 202.2 (1994): 586-605; Kool and Vlak, Arch. Virol. 130.1-2 (1993): 1-16). Furthermore, there might be time differences in PHpr and CMV promoter activity when used in the BV context. The PHpr is known to be active at very late stages in BV infection (Hasnain et al., Gene 190.1 (1997): 113-18) whereas the CMV promoter is, at least in mammalian cells, supposed to be active at very early stages of infection. Although the CMV promoter has been used in insect cells, its precise time of activity within the BV system has not yet been determined. The present results support that both transgene cassettes can be arranged in the described configuration suggesting at least a partial non-interfering activity of both promoters. The qualitative superiority in protein expression with the opposite orientations may be attributable to putative local genomic promoter interference, where a greater distance between both promoters allows a more independent expression of both transgene cassettes, although this is offered only by way of possible explanation of an underlying mechanism related to the invention and is not intended as a limitation on the invention.

The picornavirus literature indicates that VP0 processing into VP2 and VP4 requires viral RNA encapsidation (Palmenberg, Annu Rev. Microbiol. 44 (1990): 603-23; Krausslich et al., Biochimie 70.1 (1988): 119-30). In addition to the dominant 35 kDa VP0 capsid protein subunit, further Mab 10F0-reactive, ca. 28 kDa EV71-specific capsid proteins were observed in BV-infected cells comparable in size to EV71 VP2. The VP2-like proteins apparently consisting of a mix of two approximately equal-sized molecules were generally detectable in lysates of EV71 transgenic BVs when the levels of EV71 protein expression were high, e.g., at late times after infection (labeled with black arrows in FIG. 3). It is likely that the 28 kDa proteins are amino- or carboxy-terminal cleavage products of PV0. However, other possibilities are that, in contrast to the authentic and completely cytoplasmic EV71 replication cycle, a splicing event leading to the synthesis and translation of an mRNA coding for the 28 kDa proteins may occur in BV-infected insect cells. Alternatively, it is possible that the BrCr-TR 3CD protease recognizes the Chinese P1 VP4-VP2 boundary as a substrate. Such additional EV71 capsid protein subunits accumulated only when EV71 P1 and 3CD were co-expressed in BV-infected cells. However, in a transient 3CD-deficient 293TT expression system such additional 10F0-reactive VP2-specific proteins were detected comparable in size to those observed in the BV-infected insect cells. These additional VP2 capsid proteins do not appear after transient expression of the EV71 BrCr VP0 ORF in 293TT cells if the putative VP0 cleavage site is mutated. Thus, the picornavirus VP0 maturation cleavage into VP2 and VP4 may also occur without encapsidation of viral genomic RNA into a preformed capsid and even if only the VP0 subunit itself is present in a cell.

Additional 10F0-reactive proteins were detectable in lysates of BV-infected cells (FIG. 3, labeled with asterisks). According to their molecular weights these might be either PV0-VP3 (61.8 kDa) or VP2-VP3 (54.4 kDa) molecules which have not been fully processed. Such intermediate products were also detected in fractions of VLP preparations after ultracentrifugation implying that such incompletely processed molecules are part of immature VLPs. Apparently, only one of the bands of the observed triplet in the VLP preparation reacted with Mab 10F0 making it likely that one of the other bands might be unprocessed VP3-VP1. Alternatively, because there are several AUGs in the P1 ORF, cryptic translation initiation from within the P1 coding sequence in infected insect cells might lead to the synthesis of smaller EV71 capsid proteins. The observed bands are more likely incompletely processed P1 products because comparable bands have been observed in poliovirus vaccines derived from inactivated attenuated virus (Bakker et al., Vaccine 29.41 (2011): 7188-96). Interestingly, the aforementioned VP2-sized protein was also detected in EV71 VLP preparations. That makes it likely that the final proteolytic VP0 processing step into VP2 and VP4 observed in lysates of infected insect cells and in 3CD-deficient 293TT cells after expression of VP0 also leads to mature VLPs containing processed VP2 and VP4 even in the absence of encapsidated viral genomic RNA. At least a certain percentage of EV71 VLPs would therefore even more resemble fully processed infectious viral capsids. The shape of a fully matured VLP would more closely resemble an infectious viral particle and could therefore trigger a protective vaccine-based immune response more efficiently and more specifically to viral capsid protein domains suggesting a qualitative difference in protective immunity depending on the amount of fully processed mature EV71 VLPs.

For poliovirus it has been shown that the final maturation VP0 cleavage step stabilizes the virion (Hellen and Wimmer, Virology 187.2 (1992): 391-97), implying that mature EV71 VLPs with VP0 processed into VP4 and VP4 might lead to more stable molecules when produced as a vaccine. Chung et al., World J. Gastroenterol. 12.6 (2006): 921-927, did not report additional immature VLP-specific bands nor did they detect VP2 in their VLP fabrication when infected Sf9 cells were the basis for their VLP production. However, they used the P1 gene derived from the neu strain, and they had additional purification steps for obtaining EV71 VLPs. It is difficult, however, to interpret the quality of their VLP yield and purity because in their analysis the VP1 protein (39 kDa) is bigger than the VP0 protein (36 kDa), yet VP0 (35.2 kDa) should be bigger than VP1 (32.6 kDa), even in the case of the EV71 neu strain used by these investigators (http://www.ncbi.nlm.nih.gov/nuccore/AF119795).

The overall purity observed for the EV71 VLPs seems reasonably high (FIG. 6, left part, lane 3) given the fact that purification included only one ultracentrifugation step followed by dialysis. The additional bands that were observed may disappear when applying further purification steps. However, in light of the presence of additional bands in the poliovirus vaccine (Bakker et al., Vaccine 29.41 (2011): 7188-96), which has been very highly purified, this seems unlikely. The additional EV71-specific bands were also observed in all fractions containing VP0 to VP3 implying that they are co-migrating with VLPs independent from whether sucrose-based or iodixanol-based separation methods were applied. Thus it is very likely that these bands indicate the presence of immature EV71 VLPs.

The EV71 VLPs were harvested from 3×10⁷ Sf21 cells in 60 ml insect cell medium from plastic dishes as starting material three days post infection. Given the sum of the band intensities of PV0, VP1, VP2 and VP3 equals 10 μg of BSA (FIG. 6) and the relative amount of VLPs that were analyzed by SDS-PAGE, approximately 100 μg of VLPs were obtained which would be comparable to the yield of 10 mg per 10⁹ Sf9 cells (Chung et al., World J. Gastroenterol. 12.6 (2006): 921-27). EV71 VLP production process evaluation done by this group involved the construction of P1/3CD double-transgenic BVs yet with different orientations and submodules compared to the constructs of the present invention plus the stirrer-based large-scale production including several biochemical optimization details like the concentration of dissolved oxygen (Chung et al., Vaccine 28.43 (2010): 6951-57). Interestingly, when adjusting their VLP production process they used the supernatant of infected cells as a starting material altogether leading to very high VLP yields.

Different codon usage has been described in the case of the BV polyhedrin gene which is expressed very late during the infection cycle from PHpr and to very high amounts. Ranjan and Hasnain, Indian J. Biochem. Biophys. 32.6 (1995): 424-28. That implies a different quality of translational metabolism at late infection stages in baculovirus-infected insect cells. As PHpr was used in the present invention to control EV71 P1 capsid polyprotein expression, a codon-optimized P1 ORF was synthesized and analysed whether usage of this P1 ORF would lead to higher amounts of VLP production. However, the results indicated that at least for EV71 P1, codon-optimization does not lead to an increased transgene expression. In contrast, however, a very small insertion of 20 additional nucleotides coding for the L21 translation enhancing sequence into 5′ of the P1 ORF led to a much higher amount of processed P1 subunits, indicating that this element would facilitate exploitation of EV71 VLP production on an industrial scale.

Sf21 cells turned out to be superior to Sf9 cells for future large-scale EV71 VLP production because of a faster transgene processing. EV71 VLP production in High Five insect cells was not examined because of the latent insect alphanodavirus infection in these cells leading to particles comparable in size and shape to the EV71 VLPs (Li et al., J. Virol. 81.20 (2007): 10890-96). Additionally, BV infection increases nodavirus production in High Five cells. In this light it is difficult to interpret the electron microscopic EV71 VLP data with this cell line (Chung et al., Vaccine 28.43 (2010): 6951-57) and other attempts to use High Five as producer cell line for VLPs (Krammer et al., Mol. Biotechnol. 45.3 (2010): 226-34).

BV-Mock-infected Sf21 cells were tested for comparable issues putatively affecting VLP production or at least the electron microscopic demonstration of EV71 VLP formation and no evidence was found for any contaminating virus in the cell culture system. Thus, the biochemical optimization of VLP production will lead to production of a safe and efficient vaccine against the recent EV71 outbreaks.

Sequences of the Synthetic P1 Genes:

1. Natural P1 ORF of EV71 VLP P1 JEDAG
[SEQ ID NO: 9]
5′-ATGGGTTCGCAAGTGTCTACACAGCGCTCCGGTTCTCACGAAAACTCAAACTCAGCCACT
GAGGGTTCTACCATAAACTACACCACCATTAATTACTACAAAGACTCCTATGCTGCCACA
GCAGGCAAACAGAGTCTCAAGCAGGATCCAGACAAGTTTGCAAATCCTGTTAAAGACATC
TTCACTGAAATGGCAGCGCCACTGAAGTCCCCATCCGCTGAGGCATGTGGATACAGTGAT
CGAGTGGCGCAATTAACTATTGGCAACTCCACCATCACCACGCAAGAAGCGGCTAATATC
ATAGTCGGTTATGGTGAGTGGCCTTCCTACTGCTCAGATTCTGACGCTACAGCAGTGGAT
AAACCAACGCGCCCGGATGTTTCAGTGAACAGGTTTTACACATTGGACACTAAATTGTGG
GAGAAATCGTCCAAGGGATGGTACTGGAAGTTCCCGGATGTGTTAACTGAAACTGGGGTT
TTTGGGCAAAATGCACAATTCCACTACCTCTACCGATCAGGGTTCTGCATCCACGTGCAG
TGCAATGCCAGTAAATTCCACCAAGGAGCACTCCTAGTCGCTGTCCTACCAGAGTATGTC
ATTGGGACAGTGGCAGGCGGTACAGGGACGGAAGATACCCACCCCCCTTACAAGCAGACT
CAACCCGGCGCCGATGGCTTCGAGTTGCAACACCCGTACGTGCTTGATGCTGGCATCCCA
ATATCACAGTTAACAGTGTGCCCACACCAGTGGATTAATTTGAGGACCAACAACTGTGCT
ACAATAATAGTGCCATACATTAACGCACTGCCTTTTGATTCTGCCTTGAACCATTGCAAC
TTTGGCCTGTTGGTTGTGCCTATTAGCCCACTAGACTACGACCAAGGAGCGACGCCAGTA
ATCCCTATAACTATCACATTGGCCCCAATGTGTTCTGAATTCGCAGGTCTTAGGCAGGCA
GTCACGCAAGGGTTCCCCACCGAGCTAAAACCTGGCACAAATCAATTTTTAACCACCGAT
GATGGCGTTTCAGCACCTATTCTACCGAACTTCCACCCCACCCCGTGTATCCACATACCT
GGTGAAGTTAGGAACTTGCTAGAGTTATGCCAGGTGGAGACCATTCTGGAGGTTAACAAT
GTGCCCACGAATGCCACTAGCTTAATGGAGAGACTGCGCTTCCCGGTCTCAGCACAAGCA
GGGAAAGGTGAGCTGTGTGCGGTGTTTAGAGCCGATCCTGGGCGAAATGGACCATGGCAA
TCCACCTTACTGGGTCAGTTGTGCGGGTACTACACCCAATGGTCAGGATCATTGGAAGTC
ACCTTCATGTTTACTGGATCCTTCATGGCTACCGGCAAGATGCTCATAGCCTATACACCG
CCAGGAGGTCCTCTGCCCAAGGACCGGGCGACCGCCATGTTGGGCACGCACGTCATCTGG
GATTTTGGGCTGCAATCGTCTGTTACCCTTGTAATACCATGGATCAGCAACACTCATTAT
AGAGCACATGCCCGAGATGGAGTGTTTGACTACTACACCACAGGGTTAGTCAGTATATGG
TATCAGACAAATTACGTGGTTCCAATCGGTGCGCCCAACACAGCCTATATAATAGCACTA
GCGGCAGCCCAAAAGAACTTCACTATGAAATTGTGCAAGGATGCTAGTGATATCCTGCAG
ACGGGCACCATCCAGGGAGATAGGGTGGCAGATGTAATTGAAAGTTCCATAGGAGATAGC
GTGAGCAGAGCCCTCACTCACGCTCTACCAGCACCCACAGGCCAGAACACACAGGTGAGC
AGTCATCGACTGGATACAGGCAAGGTTCCAGCACTCCAAGCTGCTGAAATTGGAGCATCA
TCAAATGCTAGTGACGAGAGCATGATTGAGACACGCTGTGTTCTTAACTCGCACAGTACA
GCTGAGACCACTCTTGATAGTTTCTTCAGCAGGGCGGGATTAGTTGGAGAGATAGATCTC
CCTCTTAAGGGCACAACTAACCCAAATGGTTATGCCAACTGGGACATAGACATAACAGGT
TACGCGCAAATGCGTAGAAAGGTAGAGCTATTCACCTACATGCGCTTTGATGCAGAGTTC
ACTTTTGTTGCGTGCACACCCACCGGGGAAGTTGTCCCACAATTGCTCCAATATATGTTT
GTGCCACCTGGAGCCCCTAAGCCAGATTCTAGGGAATCCCTTGCATGGCAAACCGCCACT
AACCCCTCAGTTTTTGTCAAGCTGTCAGACCCTCCAGCGCAGGTTTCAGTGCCATTCATG
TCACCTGCGAGTGCTTATCAATGGTTTTATGACGGATATCCCACATTCGGAGAACACAAA
CAGGAGAAAGATCTTGAATACGGGGCATGTCCTAATAACATGATGGGCACGTTCTCAGTG
CGGACTGTGGGGACCTCCAAGTCTAAGTACCCTTTAGTGGTTAGGATTTACATGAGGATG
AAGCACGTCAGGGCGTGGATACCTCGCCCGATGCGTAACCAGAACTACCTATTCAAAGCC
AACCCAAATTATGCTGGCAACTCCATTAAGCCAACTGGTGCCAGTCGCACAGCGATCACC
ACTCTTTAG-3′
coding for the following amino acid sequence:
[SEQ ID NO: 10]
NH2-MGSQVSTQRSGSHENSNSATEGSTINYTTINYYKDSYAATAGKQSLKQDPDKFANPVKDI
FTEMAAPLKSPSAEACGYSDRVAQLTIGNSTITTQEAANIIVGYGEWPSYCSDSDATAVD
KPTRPDVSVNRFYTLDTKLWEKSSKGWYWKFPDVLTETGVFGQNAQFHYLYRSGFCIHVQ
CNASKFHQGALLVAVLPEYVIGTVAGGTGTEDTHPPYKQTQPGADGFELQHPYVLDAGIP
ISQLTVCPHQWINLRTNNCATIIVPYINALPFDSALNHCNFGLLVVPISPLDYDQGATPV
IPITITLAPMCSEFAGLRQAVTQGFPTELKPGTNQFLTTDDGVSAPILPNFHPTPCIHIP
GEVRNLLELCQVETILEVNNVPTNATSLMERLRFPVSAQAGKGELCAVFRADPGRNGPWQ
STLLGQLCGYYTQWSGSLEVTFMFTGSFMATGKMLIAYTPPGGPLPKDRATAMLGTHVIW
DFGLQSSVTLVIPWISNTHYRAHARDGVFDYYTTGLVSIWYQTNYVVPIGAPNTAYIIAL
AAAQKNFTMKLCKDASDILQTGTIQGDRVADVIESSIGDSVSRALTHALPAPTGQNTQVS
SHRLDTGKVPALQAAEIGASSNASDESMIETRCVLNSHSTAETTLDSFFSRAGLVGEIDL
PLKGTTNPNGYANWDIDITGYAQMRRKVELFTYMRFDAEFTFVACTPTGEVVPQLLQYMF
VPPGAPKPDSRESLAWQTATNPSVFVKLSDPPAQVSVPFMSPASAYQWFYDGYPTFGEHK
QEKDLEYGACPNNMMGTFSVRTVGTSKSKYPLVVRIYMRMKHVRAWIPRPMRNQNYLFKA
NPNYAGNSIKPTGASRTAITTL-COOH
2. Codon-optimized for insect Sf9 cells and BV expression
[SEQ ID NO: 11]
5′-ATGGGATCCCAGGTGAGTACACAGCGCTCCGGATCCCACGAAAATAGCAACTCCGCAACA
GAGGGATCTACTATAAACTATACGACCATCAACTACTATAAGGACTCATACGCCGCAACA
GCAGGTAAACAATCCTTGAAGCAGGATCCTGACAAGTTCGCGAATCCTGTTAAGGATATA
TTCACCGAAATGGCGGCTCCGCTGAAGTCGCCCTCCGCTGAAGCTTGCGGTTATAGTGAT
CGTGTAGCGCAATTGACCATTGGCAACTCTACCATCACAACACAGGAAGCCGCAAACATC
ATCGTGGGATACGGAGAGTGGCCCTCGTATTGCTCTGACTCTGACGCTACTGCGGTTGAC
AAGCCAACTCGTCCAGACGTTTCGGTGAACAGGTTCTACACCTTGGATACCAAGCTCTGG
GAGAAGAGCAGCAAAGGTTGGTACTGGAAGTTTCCTGATGTCCTGACAGAAACTGGTGTT
TTCGGACAGAACGCCCAGTTTCACTACCTGTATCGTTCCGGCTTTTGTATCCATGTGCAG
TGCAACGCATCAAAGTTCCACCAGGGAGCTCTTTTGGTGGCCGTATTGCCCGAGTACGTG
ATCGGTACCGTTGCAGGCGGTACAGGAACCGAGGACACGCACCCGCCCTACAAACAAACG
CAACCAGGAGCGGACGGTTTCGAACTTCAACACCCCTACGTTCTCGATGCTGGCATTCCT
ATCTCACAGCTGACCGTTTGCCCACACCAATGGATCAACCTCCGCACTAACAACTGCGCT
ACTATTATTGTCCCTTACATTAACGCACTCCCTTTTGACAGTGCCCTCAACCACTGTAAC
TTCGGATTGCTTGTGGTCCCTATATCCCCCCTGGACTACGACCAGGGTGCAACTCCAGTG
ATCCCTATTACGATCACCCTCGCCCCGATGTGTTCTGAGTTTGCAGGCCTTCGCCAGGCG
GTGACGCAGGGCTTCCCTACGGAGTTGAAGCCCGGCACCAACCAGTTCCTTACGACGGAC
GACGGTGTTTCTGCACCCATTCTTCCAAACTTTCATCCCACTCCATGCATCCATATACCT
GGAGAAGTTCGTAACTTGCTGGAGCTGTGCCAGGTTGAAACGATCCTCGAGGTCAATAAC
GTCCCAACTAACGCCACCAGCTTGATGGAAAGACTCAGATTCCCAGTGAGCGCCCAGGCG
GGAAAGGGAGAGCTGTGCGCTGTGTTTCGCGCTGACCCTGGACGCAACGGTCCATGGCAA
AGCACGCTCTTGGGTCAGTTGTGCGGTTACTATACTCAATGGTCAGGATCTCTGGAAGTC
ACTTTCATGTTCACTGGCAGTTTCATGGCAACTGGTAAAATGCTGATTGCTTACACACCC
CCAGGCGGACCTCTGCCAAAGGACCGTGCTACGGCCATGCTGGGCACTCACGTGATTTGG
GATTTCGGTCTCCAATCATCTGTTACGTTGGTGATTCCTTGGATCTCCAACACGCACTAT
CGCGCCCATGCACGCGACGGTGTGTTTGATTATTATACGACCGGCCTGGTTTCAATCTGG
TACCAGACCAACTATGTTGTTCCGATAGGCGCCCCAAATACGGCGTATATCATTGCCCTG
GCCGCTGCTCAAAAGAATTTCACCATGAAGCTGTGCAAGGATGCGAGCGACATCCTGCAG
ACCGGTACTATCCAGGGAGACAGGGTGGCTGACGTAATCGAGAGTTCTATAGGTGACTCA
GTTTCACGCGCTCTCACGCACGCGCTTCCTGCTCCTACCGGCCAAAACACACAAGTGAGC
TCTCATAGGCTGGACACCGGCAAGGTCCCCGCTCTGCAGGCTGCAGAGATCGGCGCCTCT
TCGAACGCATCGGATGAATCTATGATCGAAACACGTTGCGTGCTTAACTCGCACTCAACA
GCAGAGACTACGCTCGACTCCTTCTTCTCGAGGGCGGGTTTGGTTGGAGAGATTGACCTC
CCATTGAAGGGTACGACAAACCCCAACGGCTACGCGAACTGGGATATTGACATAACCGGT
TACGCCCAAATGAGAAGGAAAGTCGAGTTGTTCACATACATGAGGTTTGATGCTGAGTTT
ACTTTCGTGGCTTGCACGCCCACCGGTGAAGTCGTGCCACAGCTCCTCCAATATATGTTT
GTCCCGCCCGGTGCCCCGAAGCCCGATTCCCGCGAATCCCTGGCTTGGCAAACCGCTACG
AACCCCTCGGTCTTTGTGAAACTCTCCGACCCTCCTGCGCAAGTGTCAGTGCCTTTTATG
AGTCCTGCCAGTGCTTATCAGTGGTTCTACGATGGTTACCCGACTTTCGGCGAGCACAAG
CAAGAGAAAGATCTCGAATACGGAGCTTGTCCAAACAACATGATGGGTACATTCTCCGTA
AGGACCGTGGGAACGTCGAAGTCGAAGTATCCTCTTGTCGTCCGCATCTATATGAGAATG
AAACACGTTCGCGCTTGGATTCCGCGTCCCATGCGTAATCAAAATTACCTGTTCAAAGCC
AACCCCAACTACGCTGGTAACTCAATAAAGCCCACAGGTGCAAGTCGCACCGCAATTACT
ACCTTGTAG-3′

While illustrative embodiments have been illustrated and described, it will be appreciated that various changes can be made therein without departing from the spirit and scope of the invention.

1. A vaccine for the prevention of human EV71 disease or infection, which vaccine comprises VLPs of EV71 strain EU703812.

2. The vaccine according to claim 1, wherein the EV71 strain EU703812 VLPs comprise capsid proteins PV0, VP4, VP2, VP3 and VP1.

3. The vaccine according to claim 2, wherein the vaccine is free of infectious genomic EV71 RNA.

4. The vaccine according to claim 3, wherein the vaccine is substantially free of any RNA.

5. The vaccine according to claim 1, in a concentration of 20 to 100 μg per human dose, together with an adjuvant.

6. (canceled)

7. (canceled)

8. (canceled)

9. (canceled)

10. A method for producing a vaccine against EV71 infection or disease, the method comprising:
infecting Spodoptera frugiperda (Sf) cells with a recombinant baculovirus comprising a P1 gene of EV71 strain EU703812 and a 3CD protease gene of prototype EV71 strain BrCr-Tr under control of a CMV promoter in a bicistronic configuration,
culturing said infected Sf cells under conditions which permit expression of the EV71 genes and assembly of EV71 VLPs,
harvesting the EV71 VLPs from the culture, and
combining the EV71 VLPs with an adjuvant to produce said vaccine.

11. (canceled)

12. (canceled)

13. The method for producing a vaccine according to claim 10, wherein the P1 gene is under control of a polyhedrin promoter and the 3CD protease gene is under control of a CMV promoter.

14. The method for producing a vaccine according to claim 10, wherein the Sf cells are Sf21.

15. The method for producing a vaccine according to claim 10, wherein the vaccine is free of infectious genomic EV71 RNA.

16. A method for producing a vaccine against EV71, the method comprising:
obtaining EV71 VLPs from a culture of Sf cells infected with a recombinant baculovirus comprising a P1 gene of EV71 strain EU703812 and a 3CD protease gene of prototype EV71 strain BrCr-Tr under control of a CMV promoter in a bicistronic configuration, said infected Sf cells having been cultured under conditions which permitted expression of the EV71 genes and assembly of EV71 VLPs, and
combining the EV71 VLPs with an adjuvant to produce said vaccine against EV71.

17. A method for producing EV71 VLPs, the method comprising:
infecting a Spodoptera frugiperda (Sf) cell with a recombinant baculovirus which comprises a P1 gene of EV71 and a 3CD protease gene of EV71 under control of a CMV promoter in a bicistronic configuration, and
culturing said infected Sf cells under conditions which permit expression of the EV71 genes and assembly of EV71 VLPs.

18. The method according to claim 17, wherein the P1 gene is from EV71 strain EU703812.

19. (canceled)

20. The method according to claim 17, wherein translational enhancer L21 is inserted 5′ of the P1 gene.

21. The method according to claim 17, wherein the Sf strain is Sf21.

22. A method for preventing human enterovirus 71 (EV71) related disease or infection, the method comprising administering to an individual an effective amount of a EV71 vaccine comprising EV71 virus-like particles (VLPs) of strain EU703812 together with a pharmaceutically acceptable excipient in an amount sufficient to generate an immune response in the individual that prevents EV71 infection or disease.

23. The method of claim 22, wherein the EV71 vaccine is administered in two consecutive doses consisting of a first dose and a second dose.

24. The method according to claim 23, wherein the second dose is administered at least two months after the first dose.

25. (canceled)

26. The method according to claim 22, wherein the EV71 vaccine further comprises an adjuvant.

27. (canceled)

28. (canceled)

28. The method according to claim 22, wherein the EV71 VLPs comprise capsid proteins PV0, VP3 and VP1 or VP4, VP2, VP3 and VP1 and are free of genomic EV71 RNA.

29. The method according to claim 22, wherein strain EU703812 is the only EV71 strain present in the vaccine.

30. (canceled)
